(19) United States
(12) Patent Application Publication
(10) Pub. No.: US 2013/0303934 A1
(43) Pub. Date: Nov. 14, 2013

(54) BRAINAVATAR

(71) Applicant: Thomas F. Collura, Chagrin Falls, OH (US)

(72) Inventor: Thomas F. Collura, Chagrin Falls, OH (US)

(21) Appl. No.: 13/669,049

(22) Filed: Nov. 5, 2012

Related U.S. Application Data
(60) Provisional application No. 61/555,058, filed on Nov. 3, 2011.

(51) Int. Cl. A61B 5/0482

(52) U.S. Cl. CPC A61B 5/0482
USPC 600/545

(57) ABSTRACT

The BrainAvatar of the present invention is a combination of hardware, software and data communications devices used to implement and integrate a full featured electroencephalogram (EEG) based neurofeedback training system. The BrainAvatar is a single platform for blending EEG, Live Z-score Training (LZT), MINI-Q, whole head QEEG, mapping and all types of neurofeedback. Patient evaluation, assessment and training can now be done in a single integrated system that supports industry standards.

### RELATED APPLICATIONS

This application claims priority to U.S. Provisional Patent Application No. 61/555,058, filed on Nov. 3, 2011, a copy of which is incorporated herein by reference in its entirety.

### FIELD OF THE INVENTION

The present invention is in the field of electroencephalogram (EEG) and neurofeedback hardware and software systems and related methods of neurofeedback administration and analysis.

### SUMMARY OF THE INVENTION

The BrainAvatar of the present invention is a combination of hardware, software and data communications devices used to implement and integrate a full featured electroencephalogram (EEG) based neurofeedback training system. The BrainAvatar is a single platform for blending EEG, Live Z-score Training (LZT), MINI-Q, whole head QEEG, mapping and all types of neurofeedback. Patient evaluation, assessment and training can now be done in a single integrated system that supports industry standards.

### DESCRIPTION OF THE DRAWINGS

FIG. 1 is a 3-D Thermometer display.

FIG. 2 is a Live Map (with Z-Scores and Z-Plus options)

FIG. 3 is a 3-D Waterfall display (with training option)

FIG. 4 is an opaque scalp view

FIG. 5 is a transparent scalp view

FIG. 6 is a Montage Editor

FIG. 7 is an EEG Simulator

FIG. 8 is an EEG Selection and Marking

FIG. 9 is a Flexible Live Topographic Mapping

FIG. 10 is a Live sLORETA Projection (frontal and occipital alpha)

FIG. 11 is a Laplacian Surface Map

FIG. 12 is a screen shot of a screen and controls

FIG. 13 is a Live sLORETA Projection (inferior temporal lobe)

FIG. 14 is a Live sLORETA-Based Brain Imaging (occipital lobe)

FIG. 15 is a Live sLORETA-Based Brain Imaging (scalp 10-20 sites shown in green)

### DETAILED DESCRIPTION OF PREFERRED AND ALTERNATE EMBODIMENTS

The BrainAvatar of the present invention is a combination of hardware, software and data communications devices used to implement and integrate a full featured electroencephalogram (EEG) based neurofeedback training system. The BrainAvatar is a single platform for blending EEG, Live Z-score Training (LZT), MINI-Q, whole head QEEG, mapping and all types of neurofeedback. Patient evaluation, assessment and training can now be done in a single integrated system that supports industry standards. The integrated system includes hardware components which may be a user computer, host computer or servers, computer hard drive, Central Processing Unit (CPU), memory device, various computer peripherals such as a computer monitor, mouse, etc. and any other associated or required hardware component, as known to one having skill in the art. Other hardware components may include electrodes, contacts, switching head box, and other hardware associated with EEG-based biofeedback and neurofeedback training, as known to one having skill in the art. Data structures such as databases, arrays and other such structures may be stored in memory on the computer hardware. Various software components are used to perform various calculations, mathematical algorithms, render images and project said images onto a computer monitor or other such screen device, and other software related functions as known to one having skill in the art. Data and network communications devices may be included such as wired and/or wireless networks and any other network or communication devices known to one having skill in the art.

An EEG is the electrical activity of the brain as recorded from the head. EEG systems typically include establishing positive electrical contact with the scalp, using absorptive electrode wrap material in contact with metallic electrodes, with saturated electrode wrap material placed in contact with the scalp. Such system is described in U.S. Pat. No. 6,574,513, which is incorporated herein by reference in its entirety.

Biofeedback is the recording, monitoring and analyzing of electrical activity of the brain and a corresponding mental state of a user. A plurality of visual, auditory and/or tactile feedback mechanisms are integrated with the electrical activity of the brain to facilitate neurofeedback training of the user. The interface is provided in such a manner so as to provide the ability of the user, in the case of self-administered monitoring, or the trainer, in the case of an administered session, to record, manage and control brain activity for different purposes including self-improvement. A system and method for biofeedback administration is described in U.S. Patent Application No. 2010/0094156, which is incorporated herein by reference in its entirety.

Methods for analyzing the biofeedback data are used, such as Quantitative EEG (QEEG). QEEG includes spectral analysis and generally includes topographic mapping and normative database analysis or the measurement, using digital technology, of electrical patterns at the surface of the scalp which primarily reflect cortical electrical activity or brainwaves.

Biofeedback data and QEEG analysis is used teach or train the subject or trainee using neurofeedback. Neurofeedback is a form of biofeedback training that uses the EEG as the signal used to control feedback. Sensors applied to the trainee's scalp record the brainwaves, which are converted into feedback signals by a human/machine interface using a computer and software. By using visual, sound, or tactile feedback to produce operant conditioning of the brain, it can be used to induce brain relaxation through increasing alpha waves. A variety of additional benefits derived from the improved ability of the central nervous system to relax, may also be obtained. Neurofeedback or brain wave training is a learning tool which teaches the brain self-regulation through the use of the relaxation/concentration process. Neurofeedback is also known as brain fitness. Methods of biofeedback training are described in U.S. Patent Application Publication No. 2009/0118636, which is incorporated herein by reference in its entirety.

Using the BrainAvatar system (“the system”) of the present disclosure and related invention, a user can acquire an EEG, view assessments and reports and perform neurofeedback training in one continuous process. It is no longer necessary to stop acquiring QEEG data to perform neurofeedback and it is not necessary to stop neurofeedback training to acquire QEEG. Data assembly, assessment, training and reporting can be done in a single, integrated, coordinated process with one system that supports scientific, industry and regulatory standards.

sLORETA Export Function

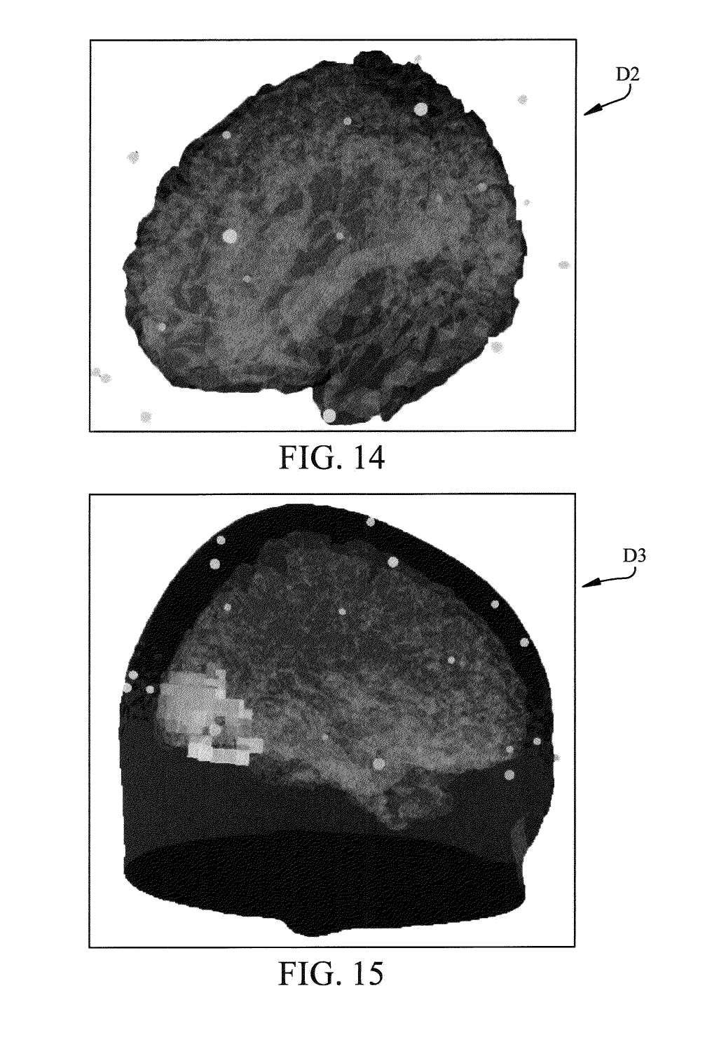

Three dimensional distribution of the electrically active neural tissue can be evaluated using standardized low resolution brain electromagnetic tomography or sLORETA. The BrainAvatar system is based on the SLORETA mathematics and theory. The system provides a LORETA export option allowing users to process EEG waveforms and export data to the LORETA for offline localization. This feature allows users to select portions of EEG, live or reviewed, process the data, and export the results to LORETA. Output can be provided in the form of raw EEG amplitude, filtered EEG (delta, theta, etc.) or slow cortical potentials (SCPs). Once exported and read into LORETA, EEG data can be imaged and analyzed using any LORETA tools and methods, as shown in FIGS. 13-15, as further described herein. FIG. 13 is a representative display D1 of a Live sLORETA Projection of the inferior temporal lobe as displayed on a representative active control screen of the neurofeedback system. FIG. 14 is a representative display D2 of an image of a Live sLORETA-based brain image of the occipital lobe, which can be displayed as an image in conjunction with the other display screens and control functions of the neurofeedback system. FIG. 15 is representative display D3 of an image of a Live sLORETA-based brain image of a scalp with multiple feedback sites graphically represented.

Live sLORETA Projector (LLP) and ZBuilder

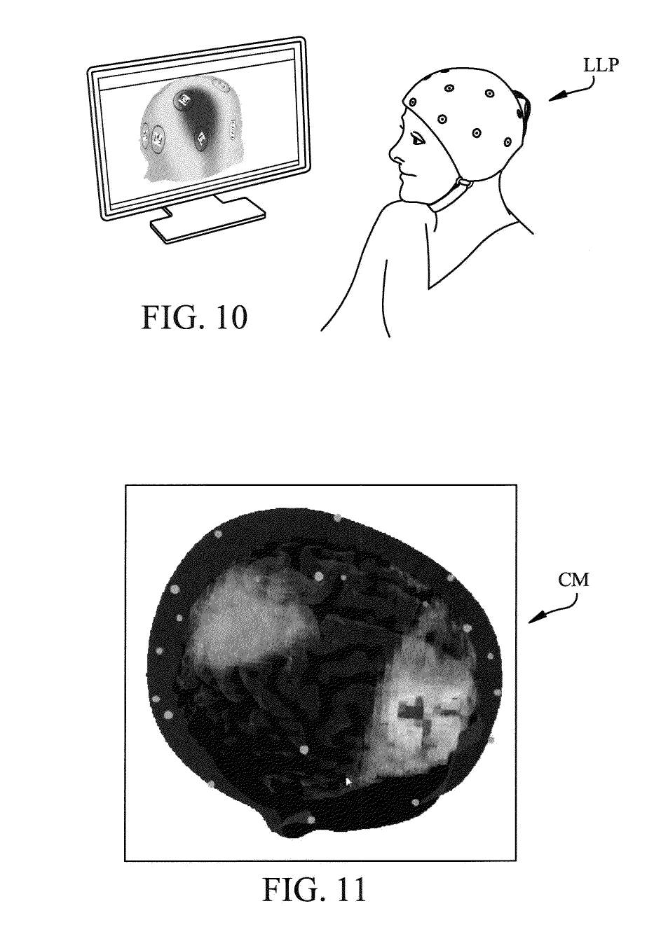

Another aspect of the BrainAvatar system is the Live sLORETA Projector (LLP) (example shown in FIG. 10), which provides live high-resolution sLORETA projections using 19 channels of EEG. The LLP provides, for the first time, live EEG-based brain functional imaging and biofeedback based on scientific principles. sLORETA is one type of transform-based brain localization technique, that is included. Other transform-based methods may also be used. It is possible to define regions of interest (ROIs) for any anatomical locations or Brodmann areas and to monitor and train EEG parameters specifically to those areas. Live scalp distributions as well as live sLORETA displays are possible during training This feature makes it unnecessary to operate the separate sLORETA software in connection with the system and allows instantaneous, live-animation representations of brain activity derived from surface EEG data. LLP can be used to train any EEG components by brain region instead of by sensor site. Any types of conventional or connectivity training can be done with LLP which makes any chose brain ROI look like an event wizard variable or EEG channel. LLP makes it possible to train brain locations specified anatomically, such as the anterior cingulate cyrus or dorsolateral frontal lobe, as well as Brodmann areas such as the visual association or speech production areas. This makes it possible to select brain structures as easily as selecting a different channel for training LLP provides a seamless real-time bridge between live EEG and brain localization, useful both with and without Z-Score training norms. LLP uses the latest 5 mm sLORETA voxel database providing over 6,000 voxels and providing accurate, high-resolution mapping of brain function in real time.

LLP uses a dipole-based modeling approach so every voxel, region of interest, or Brodmann area can be represented by an equivalent dipole, or by individual dipoles. This provides unprecedented ability to visualize brain functional sources as collections of synchronous neurons (pyramidal cells), combining anatomical with functional perspective. Introduction of dipole angular information opens the door to Joint Space-Time Frequency Analysis (JSTFA). Now, brain function in space as well as time can be monitored, assessed and trained.

ZBuilder allows users to create their own targeting templates for live Z-Score training (LZT) with or without using a normative database. In its simplest form, ZBuilder allows you to design training profiles for any of a wide range of EEG parameters including absolute and relative power, power ratios, asymmetry, coherence, phase, synchrony, spectral correlation, or co-modulation. A user may capture a client's EEG, use it to create a training template, and make changes suitable for clinical intervention. For example, if the goal is to reduce beta in the cingulate gyrus, a training template can be created that is based upon the client's own EEG parameters, modified to reduce beta in that area only. This provides the possibility for individualized training that does not depend on training for individualized training that does not depend on training everyone to the same norm. This new capability takes live Z-Score training beyond the “one size fits all” perspective and opens the door to truly individualized LZT training

ZBuilder can also be used to visualize and quantify changes in brain activation and connectivity associated with different states, including tasks, acquire a baseline EEG and use it to create a visualization template. As long as the EEG does not change, the display will be clear. As soon as a particular brain location activates, it will show up in the display as a dynamic change. This makes it possible for the first time to visualize dynamic changes in brain activity associated with various functions, for diagnostic as well as for training purposes (“digital subtraction brain electrical imaging”). Brain activity can be captured either as individual channels or as LLP regions of interest. For the first time, it is now possible to simply select a brain region, record from it, and perform brain structural operant training as easily as traditional training using individual leads. LLP training can also be combined with traditional training, allowing a flexible choice of training targets with the continual ability to visualize and measure brain activity in 3 dimensions.

With LLP, it is also possible to do LORETA-based connectivity training A user may select any two regions of interest and LLP will allow the user to monitor and train coherence, phase, spectral correlation, co-modulation, or synchrony between those sites in any selected band. Specifically target functional connections using LLP to address functional connections in a simple, intuitive, easy-to-use and understand system.

LLP and ZBuilder are integrated with an Event Wizard, allowing users to instantly integrate sLORETA-based neurofeedback, with or without Z-Scores, into any protocol. Custom Z-Score templates can be combined with traditional or peripheral biofeedback in a seamless implementation. For example, to downtrain beta in the anterior cingulate gyrus, a single event using an equation as simple as “x=ROI(ANTCING, BETA)” will provide instantaneous training data derived from this brain locus.

LLP provides the flexibility to select any subset of the standard 19 10-20 sites and use the readings to do limited localization and lateralization for special needs. For example, 4 sensors placed at F3, F4, P3 and P4 will adequately isolate activity that is arising from the cingulate area. It is possible to identify localization, anterior-posterior position, and degree of closeness to the midline using LLP and these 4 sites. While this is not intended for specific localization, as an assessment technique it has value in determining the gross location and magnitude of specific frequencies arising from these structures. LLP can also be used with any other number of sites with the resolution and accuracy depending on the sites selected. It is also possible, for example, to focus on posterior areas by recording from the rear 8 sites: Cz, Pz, P3, P4, T5, T6, O1 and O2. With LLP's live 3-D projector, it is possible to see all instantaneous sLORETA data in real time to confirm hypotheses and to guide training

### Peripherals and DC/Slow Cortical Potentials

The BrainAvatar system supports the full range of peripheral biofeedback devices including hemoencephalography (HEG), temperature (TEMP), blood-volume pulse (BVP), heart rate (HR), heart-rate variability (HRV), skin conductance response (SCR), respiration (RESP) and electromyography (EMG). Protocol and screen designs are available online to provide individual or combined biofeedback modalities, with or without concurrent EEG. Couples and healer/healee biofeedback and neurofeedback are also possible. New designs provide mind-body training, such as pioneered by Maxwell Cade, and adapted for heart/brain training using these new protocol concepts and approaches. The system provides a full set of DC and slow-cortical potential (SCP) capabilities. Bipolar infra-low frequency (ILF) training can be efficiently provided using simple designs, and combined with Live Z-Scores, peripherals, or Live LORETA training In addition, the European style of SCP training can be conducted using randomized alternating trials on monopolar EEG training, as reported from the University of Tubingen.

### Computer Displays

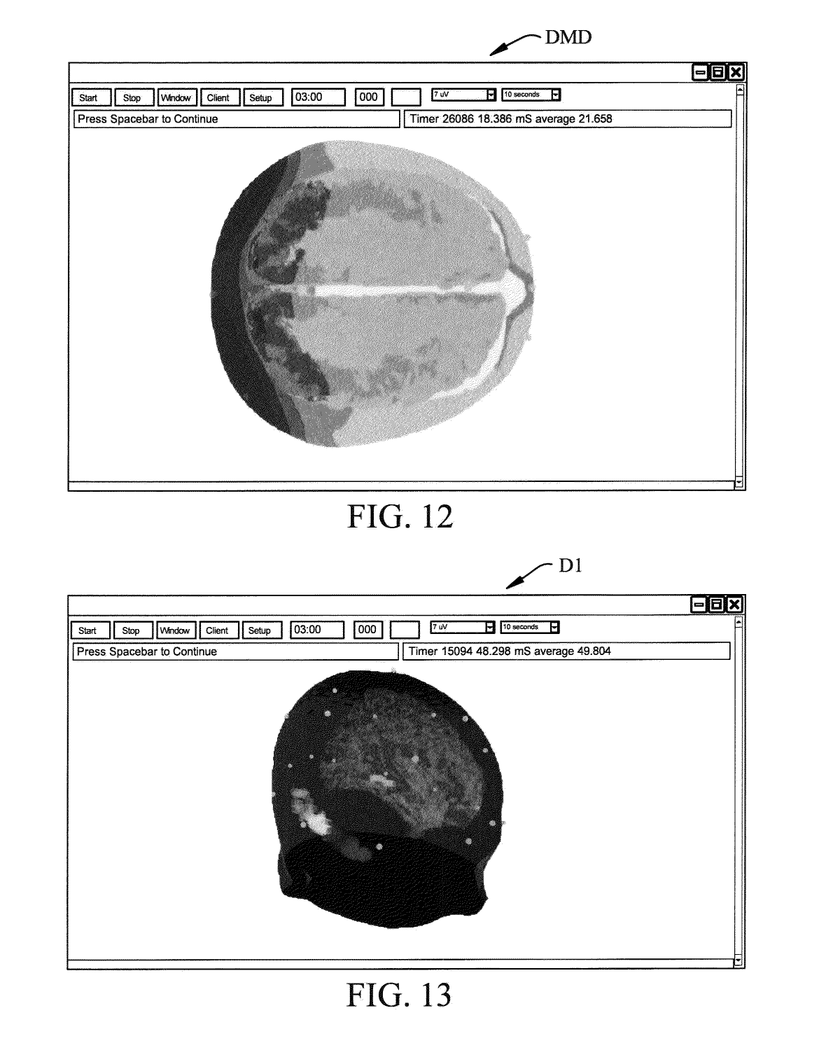

The BrainAvatar system features an innovative new dual monitor display with independent controls, a representation of which is shown as DMD in FIG. 12), with up to 8 tabbed screens on each monitor. This allows a user to select quickly between waveforms, graphs, training screens, live text, whole-head maps, and other displays. All screen scan be modified in real time, allowing users to develop and evolve the training software over time. Pull down menus simplify tailoring each display panel to suit individual user needs. All settings are automatically used in real time and are saved in client folders or standard setups.

Users can run any design from a desktop shortcut and then select a client folder or create new folders on the fly, allowing users to arrange work flow to each user's particular preference. Trainee data as well as settings files can be stored in subfolders and selected using a built-in file browser. Raw data, summaries, images or other saved data can be accessed using an intuitive and simple user interface.

The BrainAvatar screens feature programmable looks or themes, and have adjustable color, grain, scale and other settings. Panels can be set up with unique views of EEG channels, filtered and FFT data, and spectra, allowing users to set up displays with unprecedented flexibility.

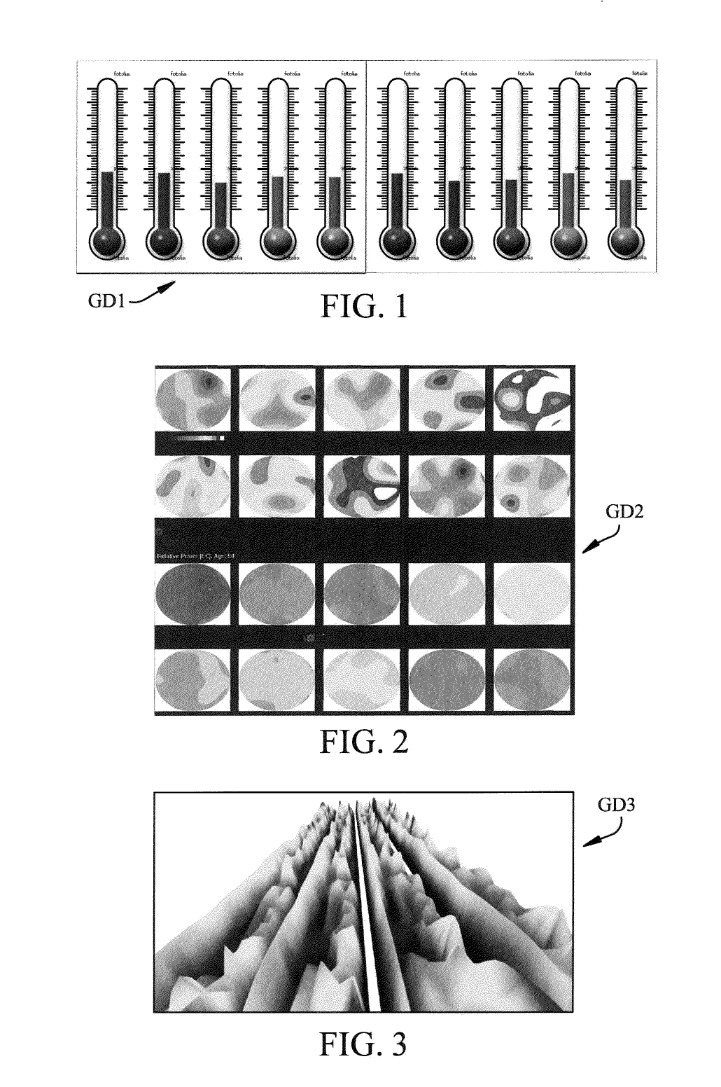

The BrainAvatar systems also adds a new set of 3-D screens, providing vivid and rapid indicators of EEG parameters and training variables. New 3-D look panels include thermometers, spectra, and other such graphical displays of various channels representing various neurofeedback signals and responses, indicated as GD1, GD2 and GD3 in FIGS. 1-3 respectively. True 3-D displays using advanced graphics techniques provide fast and visually appealing professional-quality 3-D images and animations. Using the latest software graphics technology, the BrainAvatar system includes displays including new Live-Q maps and 3-D renderings providing live parameters, Z-Scores and maps.

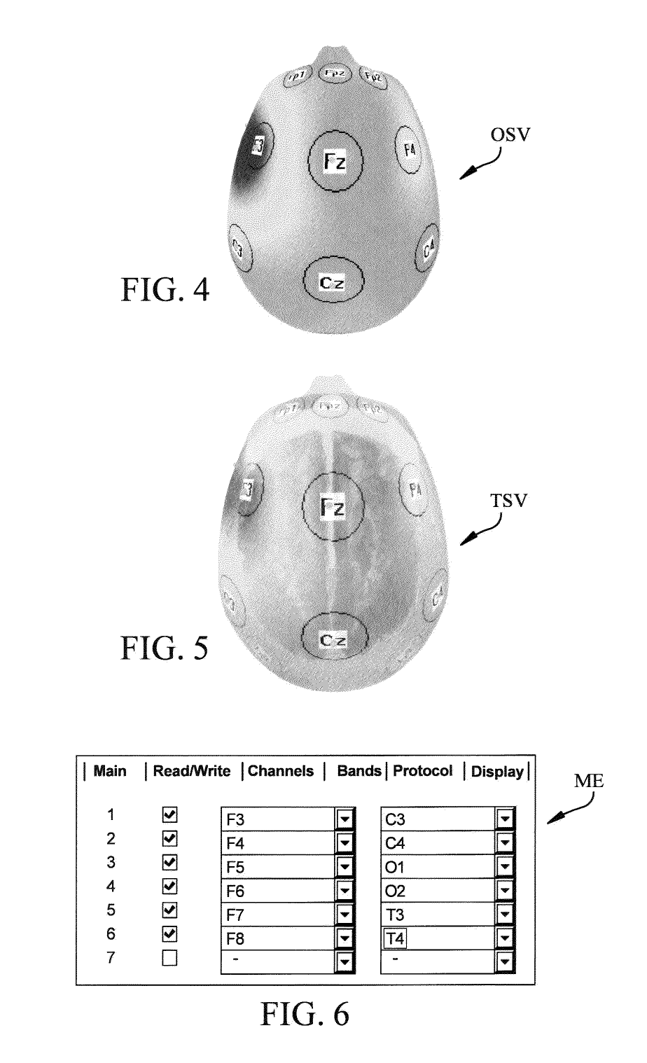

The opaque scalp view, an example of which is indicated as OSV in FIG. 4, shows EEG surface distribution in realistic coordinates. All views can be easily rotated and sized to suit individual user needs. The transparent scalp view, an example of which is indicated as TSV in FIG. 5, shows underlying brain structures in accurate LORETA coordinates.

Live-Q displays reflect the brain's dynamics in real time, with unprecedented detail and speed. This leads to faster, more accurate assessments, and targeted neurofeedback training using the latest Live Z-Score training including the exclusive PZOK and ZPLUS training capabilities. These live maps show brain activity in real time, showing brain activity as raw data or as referenced to a normative database.

The system also includes simple flexible tools to reformat, edit, mark, and artifact EEG recordings. A montage editor, an example of which is indicated as ME in FIG. 6, is configured to enable creation and use of montages, and to quickly switch to different montages on-the-fly, or during file review.

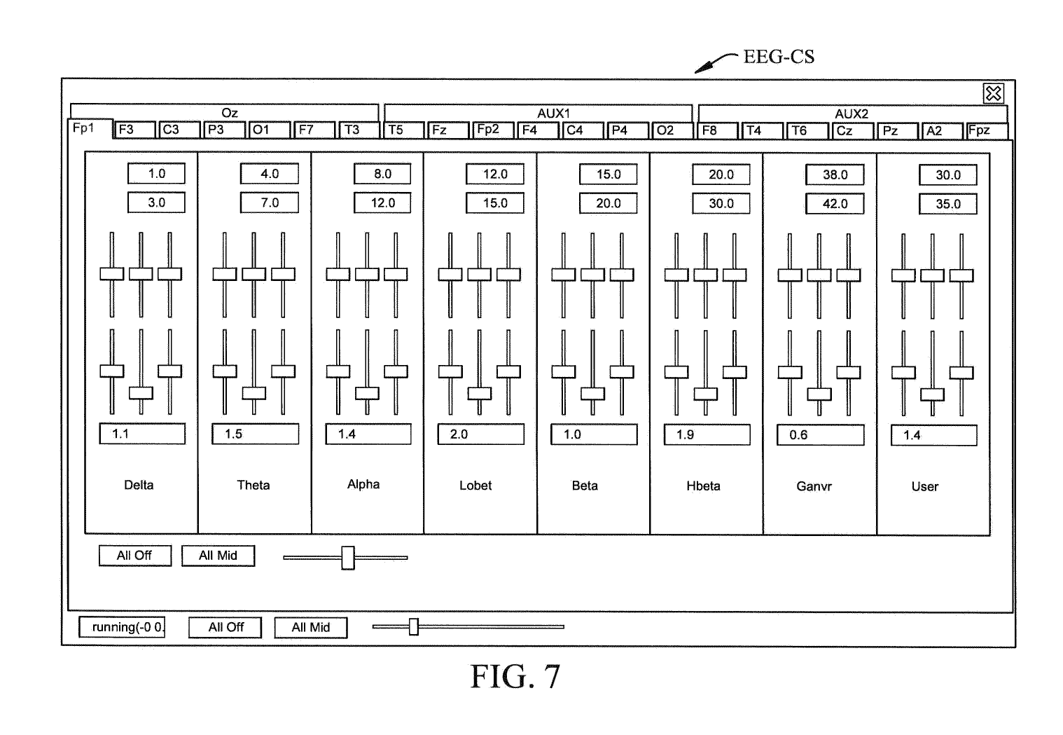

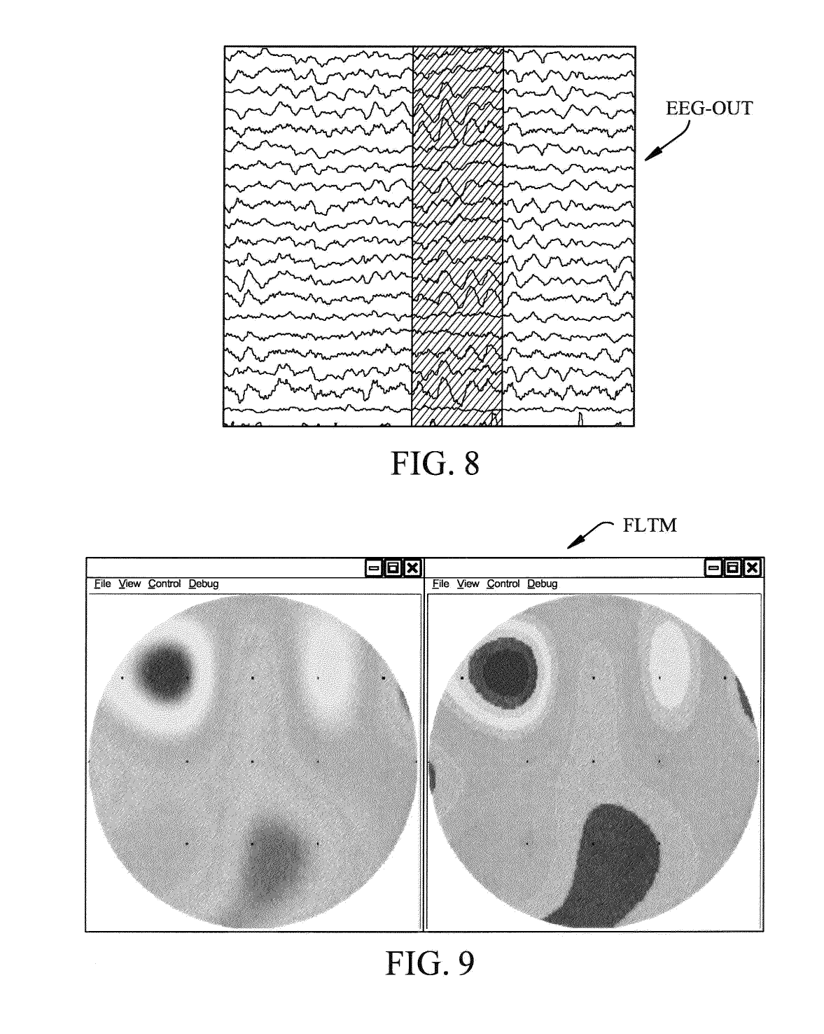

The BrainAvatar system includes a built-in EEG control simulator or dashboard or virtual control panel, a representative embodiment of which is shown in FIG. 7, by which EEG channels and records can be selected and recorded, as shown in the example output EEG-out shown in FIG. 8, that can be used for teaching, testing, validation, or sham (mock) biofeedback applications. It features 8 bands of independently controlled non-linear coupled oscillators, with programmable amplitude, range, and variability. Along with live EEG, simulated data can be rendered into graphics, 2-d and 3-D displays, and summary files for statistical processing and Z-Score computations.

The new screens allow practitioners to assess and visualize brain dynamics in space and time, showing realistic and meaningful representations of dynamic properties such as attraction, repulsion, gobal size, and tendency for self-organization. You can now visualize the change, motivation, and resultant self-efficacy evident in QEEG parameters including Z-Scores, in real time and during other clinical interventions.

With the BrainAvatar system, users can alternate and combine EEG with 2-D and 3-D views, contour maps CM, an example of which is shown in FIG. 11, and brain source images. The system enables users to view from any angle, change background, and use transparent or opaque images to view the brain and scalp EEG individually or together, and to produce flexible live topographic imaging and marking FLTM as shown in FIG. 9 on one or multiple displays. Any component including Z-Scores, monopolar, or sequential data can be viewed. References include linked ears, individual ears, average, Laplacian, and LORETA localization data. Selectable rendering and smoothing parameters let you tailor maps and images to your needs based on your preference of quantization, color scheme, and model.

With a new screen saver feature users may print or save BrainAvatar screens to disk, at any time at the press of a button. Screens can also be instantly printed for rapid availability, even. during the session. Screen images are automatically numbered and can be labeled, printed, or imported into documents, spreadsheets, or reports.

New screen displays carry forward to home, school, office, and other remote environments. Clinicians can select the amount of control to give remote users and set limits by number of sessions, by training time, or by calendar. By locking down the settings, providers can ensure that clients have a uniform experience and comply with time and session requirements. Improved ease of use for remote supervision includes faster access to on-screen controls when a clinician is supervising a live client over the Internet.

What is claimed is:

1. An EEG-based neurofeedback training system comprising:
a combination of computer hardware and software operative to receive EEG data, perform assessments of said EEG data, create reports based on said EEG data; render said reports and data on a computer screen or monitor, perform neurofeedback training within a single fully-integrated EEG-based neurofeedback training system.

2. The EEG-based neurofeedback training system of claim 1, wherein the system includes user-programmable tabbed screens in which specific panels can be selected, moved, and configured for specific and changing display needs.

3. The EEG-based neurofeedback training system of claim 1, wherein the system includes 16 frequency bands that are adjustable and user-programmable and implemented in digital filters as well as via spectral analysis software, for assessment or operant training

4. The EEG-based neurofeedback training system of claim 1 further comprising a 24-channel EEG simulator for testing, research and mock feedback, providing the ability to produce specified waveforms of specified frequency, amplitude, and phase relationships, in combination with live or retrospective EEG signals.

5. The EEG-based neurofeedback training system of claim 1, wherein the system is capable of rendering 2-D live topographic maps and displaying same on a computer screen, using raw data or database-referenced live z-scores for amplitude, power, coherence, phase, asymmetry, or related measures, for assessment or operant training

6. The EEG-based neurofeedback training system of claim 1, wherein the system is capable of rendering 3-D live topographic maps and displaying same on a computer screen, reflecting dynamic patterns of brain activation and connectivity in real time, for assessment or operant training

7. The EEG-based neurofeedback training system of claim 1 further comprising instantaneous live sLORETA projector with real-time 3-D imaging, producing live images of brain activation patterns internal to the brain, at high resolution and with instantaneous response, for assessment or operant training

8. An EEG-based biofeedback system comprising:
a computer;
an EEG simulator for testing, research and feedback;
software contained on the computer operative to:
receive and store live EEG data;
use sLORETA or a related algorithm to produce and project live 3-D brain imaging
based on said EEG data onto computer screen.

9. The EEG-based biofeedback system of claim 8, wherein the system is capable of displaying editable and user-programmable live and retrospective EEG and/or peripheral physiological review screens.

10. The EEG-based biofeedback system of claim 8, wherein the system is capable of rendering contoured and 3-D display panels based on EEG data useful for assessment or operant training

11. The EEG-based biofeedback system of claim 8, wherein the system is capable of rendering user-programmable tabbed screens in which specific panels can be selected, moved, and configured for specific and changing display needs.

12. The EEG-based biofeedback system of claim 8, wherein the system is capable of displaying waveforms, graphs, training screens, live text, and whole-head maps, useful for assessement or operant training.

13. The EEG-based biofeedback system of claim 8, wherein each display screen can be modified by a user in real-time to alter its contents, scale, color, arrangement, or other related parameters relevant to assessment or operant training.

14. The EEG-based biofeedback system of claim 8, wherein the system is capable of displaying Live-Q displays accurately and rapidly reflecting the brain's dynamic patterns of activation in real time, useful for assessment or operant training.

15. The EEG-based biofeedback system of claim 8 further comprising 16 bands of independently controlled non-linear coupled oscillators, with programmable amplitude, range and variability.

16. The EEG-based biofeedback system of claim 8 further comprising a method for constructing Z-Score targets based upon EEG recordings, thus providing the means for live z-score training with user-determined and user-defined statistical criteria.
